(19) United States
(12) Patent Application Publication
(10) Pub. No.: US 2006/0030579 A1
(43) Pub. Date: Feb. 9, 2006

(54) Compounds for the treatment of proliverative processes

(75) Inventors: John Edward Park, Biberach (DE); Nveed Chaudhary, Biberach (DE); Michael P. Pieper, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/074,179

(22) Filed: Mar. 7, 2005

(30) Foreign Application Priority Data
Mar. 30, 2004 (DE) 10 2004 016 179.8

(51) Int. Cl. A61K 31/4745; A61K 31/46; A61K 31/137

(52) U.S. Cl. 514/291; 514/305; 514/649; 514/304

(57) ABSTRACT

The invention relates to the use of anticholinergics for preparing a pharmaceutical composition for the prevention and treatment of proliferative processes.

This application claims priority benefit from German application DE 10 2004 016 179.8, filed Mar. 30, 2004.

The invention relates to the use of anticholinergics for preparing a pharmaceutical composition for the prevention and treatment of proliferative processes.

### BACKGROUND TO THE INVENTION

Proliferative processes in various tissues are responsible for pathophysiological states in all kinds of diseases, including diseases of the respiratory tract. These processes lead to remodelling of the lung tissue. Thus, for example, the proliferation of fibroblasts of the bronchi and bronchioles can be observed particularly at advanced stages of disease. The changes in the vascular system of the lungs may in turn induce severe disorders in cardiac function (cor pulmonale).

The aim of the present invention is to prepare pharmaceutical compositions which counteract the proliferative processes described above.

### DESCRIPTION OF THE INVENTION

Surprisingly it has been found that the antimuscarinically active anticholinergics 1 are characterised by an anti-proliferative effect.

Accordingly, the present invention relates to the use of anticholinergics 1 for preparing a pharmaceutical composition for the prevention and treatment, particularly the treatment, of proliferative processes.

The invention further relates to the use of anticholinergics 1 for preparing a pharmaceutical composition for the prevention and treatment, particularly the treatment, of diseases associated with proliferative processes.

The long-acting anticholinergics 1 to be used within the scope of the present invention are preferably selected from the group consisting of tiotropium salts (1.1), oxitropium salts (1.2), flutropium salts (1.3), ipratropium salts (1.4), glycopyrronium salts (1.5), trospium salts (1.6) and the compounds of formulae 1.7 to 1.13.

In the above-mentioned salts 1.1 to 1.6 the cations tiotropium, oxitropium, flutropium, ipratropium, glycopyrronium and trospium denote the pharmacologically active constituents. Explicit references to the above-mentioned cations are made using the terms 1.1′ to 1.6′. Any reference to the above-mentioned salts 1.1 to 1.6 naturally also includes a reference to the corresponding cations tiotropium (1.1′), oxitropium (1.2′), flutropium (1.3′), ipratropium (1.4′), glycopyrronium (1.5′) and trospium (1.6′).

By the salts 1.1 to 1.6 are meant according to the invention those compounds which contain in addition to the cations tiotropium (1.1′), oxitropium (1.2′), flutropium (1.3′), ipratropium (1.4′), glycopyrronium (1.5′) and trospium (1.6′) chloride, bromide, iodide, sulphate, phosphate, methanesulphonate, nitrate, maleate, acetate, citrate, fumarate, tartrate, oxalate, succinate, benzoate or p-toluenesulphonate as counter-ion (anion), the preferred counter-ions being chloride, bromide, iodide, sulphate, methanesulphonate or p-toluenesulphonate. Of all the salts, the chlorides, bromides, iodide and methanesulphonate are particularly preferred.

In the case of the trospium salts (1.6) the chloride is particularly preferred. In the case of the other salts 1.1 to 1.5 the methanesulphonates and bromides are of particular significance.

Of particular importance are pharmaceutical combinations which contain tiotropium salts (1.1), oxitropium salts (1.2) or ipratropium salts (1.4), the associated bromides being of particular importance according to the invention. Tiotropium bromide (1.1) is of particular importance. The above-mentioned salts may optionally be present in the drug combinations according to the invention in the form of their solvates or hydrates, preferably in the form of their hydrates. In the case of tiotropium bromide the drug combinations according to the invention preferably contain it in the form of the crystalline tiotropium bromide monohydrate, which is known from WO 02/30928. If tiotropium bromide is used in anhydrous form in the drug combinations according to the invention, anhydrous crystalline tiotropium bromide is preferably used, which is known from WO 03/000265.

The above-mentioned anticholinergics 1 may optionally have chiral carbon centres. In this case the pharmaceutical compositions according to the invention may contain the anticholinergics in the form of their enantiomers, mixtures of the enantiomers or racemates, while enantiomerically pure anticholinergics are preferably used, such as for example in the case of the salts 1.4 and 1.5.

In another preferred embodiment of the present invention compounds of formula 1.7 are used as anticholinergics 1, 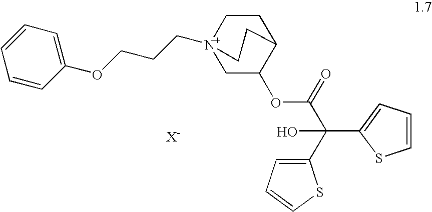
wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the racemates, enantiomers or hydrates thereof.

Preferred salts of formula 1.7 are those wherein
X⁻ denotes an anion with a single negative charge, preferably an anion selected from among the fluoride, chloride, bromide, methanesulphonate and p-toluenesulphonate, preferably bromide,
   optionally in the form of the racemates, enantiomers or hydrates thereof.

Also preferred are salts of formula 1.7 wherein
X⁻ denotes an anion with a single negative charge, preferably an anion selected from among the chloride, bromide and methanesulphonate, preferably bromide,
   optionally in the form of the racemates, enantiomers or hydrates thereof.

Particularly preferred are compounds of formula 1.7 in the form of the bromides.

Of particular importance are those compounds of formula 1.7 which contain the enantiomers of formula 1.7-en 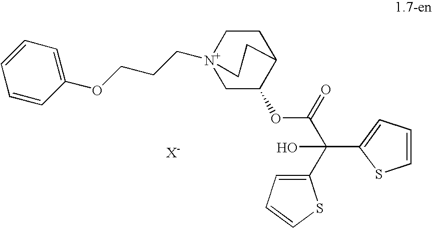
wherein X⁻ may have the above-mentioned meanings.

In another preferred embodiment of the present invention compounds of formula 1.8 are used as anticholinergics 1, 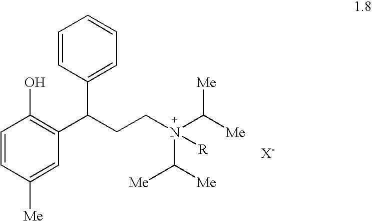
wherein R denotes either methyl (1.8.1) or ethyl (1.8.2) and wherein X⁻ may have the above-mentioned meanings. In an alternative embodiment the compound of formula 1.8 is present in the form of the free base 1.8-base

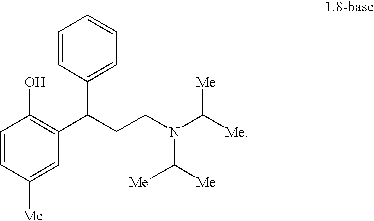

In another preferred embodiment of the present invention compounds of formula 1.9 are used as anticholinergics 1, 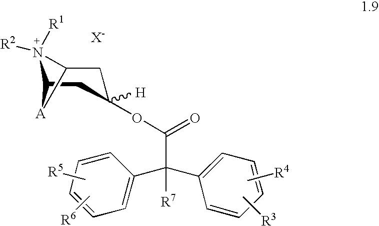
wherein
A denotes a double-bonded group selected from the groups 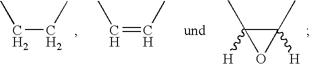
X⁻ denotes one of the above-mentioned anions with a single negative charge, preferably a chloride, bromide or methanesulphonate,
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine.

The compounds of formula 1.9 are known in the art (WO 02/32899).

Within the scope of the present invention preferred compounds of formula 1.9 are those wherein
X⁻ denotes bromide;
R¹ and R² which may be identical or different denote methyl or ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine.

Of particular importance are those compounds of formula 1.9 wherein
A denotes a double-bonded group selected from

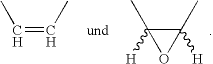

Of particular importance are the following compounds of formula 1.9:
tropenol 2,2-diphenylpropionate methobromide (1.9.1),
scopine 2,2-diphenylpropionate methobromide (1.9.2),
scopine 2-fluoro-2,2-diphenylacetate methobromide (1.9.3),
tropenol 2-fluoro-2,2-diphenylacetate methobromide (1.9.4).

The compounds of formula 1.9 may optionally be present in the form of their enantiomers, mixtures of their enantiomers or racemates, as well as optionally in the form of their hydrates and/or solvates.

In an equally preferred embodiment of the present invention compounds of formula 1.10 are used as anticholinergics 1, 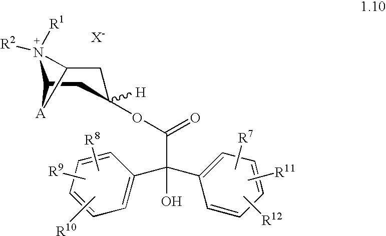
wherein
A, X, R¹ and R² may have the meanings given above and wherein R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen.

The compounds of formula 1.10 are known in the art (WO 02/32898).

Within the scope of the present invention preferred compounds of formula 1.10 are those wherein
A denotes a double-bonded group selected from 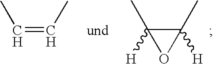
X⁻ denotes bromide;
R¹ and R2 which may be identical or different denote methyl or ethyl, preferably methyl;
R⁷, R⁸, R⁹, R¹⁰, R¹¹ and R¹², which may be identical or different, denote hydrogen, fluorine, chlorine or bromine, preferably fluorine, while at least one of the groups R⁷, R⁸, R⁹, R¹⁰, R¹¹ and R¹² may not be hydrogen.

Of particular importance are those compounds of formula 1.10 which are selected from the group consisting of:
tropenol 3,3′,4,4′-tetrafluorobenzilate methobromide (1.10.1),
scopine 3,3′,4,4′-tetrafluorobenzilate methobromide (1.10.2),
tropenol 4,4′-difluorobenzilate methobromide (1.10.3),
scopine 4,4′-difluorobenzilate methobromide (1.10.4),
tropenol 3,3′-difluorobenzilate methobromide (1.10.5),
scopine 3,3′-difluorobenzilate methobromide (1.10.6);

The compounds of formula 1.10 may optionally be present in the form of their enantiomers, mixtures of their enantiomers or racemates, as well as optionally in the form of their hydrates and/or solvates.

In another preferred embodiment of the present invention compounds of formula 1.11 are used as anticholinergics 1, 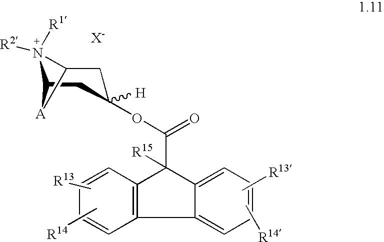
wherein
A and X⁻ may have the meanings given above and wherein
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen, or
R^{1′} and R^{2′} together denote a —C₃-C₅-alkylene bridge;
R¹³, R¹⁴, R^{13′} and R^{14′} which may be identical or different, denote hydrogen, —C₁-C₄-alkyl, —C₁-C₄-alkyloxy, hydroxy, —CF₃, —CHF₂, CN, NO₂ or halogen.

The compounds of formula 1.11 are known in the art (WO 03/064419).

Preferred compounds of formula 1.11 are those wherein
A denotes a double-bonded group selected from 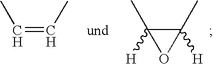
X⁻ denotes an anion selected from chloride, bromide and methanesulphonate, preferably bromide;
R¹⁵ denotes hydroxy, methyl or fluorine, preferably methyl or hydroxy;
R^{1′} and R^{2′} which may be identical or different, denote methyl or ethyl, preferably methyl;
R¹³, R¹⁴, R^{13′} and R^{14′} which may be identical or different, denote hydrogen, —CF₃, —CHF₂ or fluorine, preferably hydrogen or fluorine.

Particularly preferred compounds of formula 1.11 are those wherein
A denotes a double-bonded group selected from 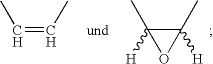
X⁻ denotes bromide;
R¹⁵ denotes hydroxy or methyl, preferably methyl;
R^{1′} and R^{2′} which may be identical or different, denote methyl or ethyl, preferably methyl;
R¹³, R¹⁴, R^{13′} and R^{14′} which may be identical or different, denote hydrogen or fluorine.

Of particular importance are compounds of formula 1.11, which are selected from the group comprising:
tropenol 9-hydroxy-fluorene-9-carboxylate methobromide (1.11.1);
tropenol 9-fluoro-fluorene-9-carboxylate methobromide (1.11.2);
scopine 9-hydroxy-fluorene-9-carboxylate methobromide (1.11.3);
scopine 9-fluoro-fluorene-9-carboxylate methobromide (1.11.4);
tropenol 9-methyl-fluorene-9-carboxylate methobromide (1.11.5);
scopine 9-methyl-fluorene-9-carboxylate methobromide (1.11.6).

The compounds of formula 1.11 may optionally be present in the form of their enantiomers, mixtures of their enantiomers or racemates, as well as optionally in the form of their hydrates and/or solvates.

In another preferred embodiment of the present invention compounds of formula 1.12 are used as anticholinergics 1, 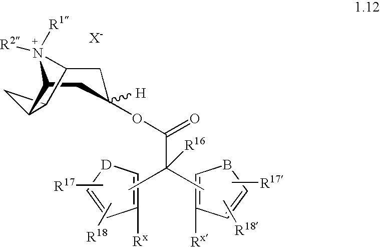
wherein X⁻ may have the meanings given above and wherein
D and B which may be identical or different, preferably identical, denote O, S, NH, CH₂, CH═CH or —N(C₁-C₄-alkyl)-;
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen, or
R^{1″} and R^{2″} together denote a —C₃-C₅-alkylene bridge; R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{X} and R^{X′} which may be identical or different, denote hydrogen, —C₁-C₄-alkyl, —C₁-C₄-alkyloxy, hydroxy, —CF₃, —CHF₂, CN, NO₂ or halogen, or
R^{X} and R^{X′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂.

The compounds of formula 1.12 are known in the art (WO 03/064418).

Preferred compounds of formula 1.12 are those wherein
X⁻ denotes chloride, bromide or methanesulphonate, preferably bromide;
D and B which may be identical or different, preferably identical, denote O, S, NH or CH═CH;
R¹⁶ denotes hydrogen, hydroxy, —C₁-C₄-alkyl, —C₁-C₄-alkyloxy, —CF₃, —CHF₂, fluorine, chlorine or bromine;
R^{1″} and R^{2″} which may be identical or different, denote C₁-C₄-alkyl, which may optionally be substituted by hydroxy, fluorine, chlorine or bromine, or
R^{1″} and R^{2″} together denote a —C₃-C₄-alkylene bridge;
R¹⁷, R¹⁸, R^{17′} and R^{18′}, which may be identical or different, denote hydrogen, C₁-C₄-alkyl, C₁-C₄-alkyloxy, hydroxy, —CF₃, —CHF₂, CN, NO₂, fluorine, chlorine or bromine;
R^{X} and R^{X′} which may be identical or different, denote hydrogen, C₁-C₄-alkyl, C₁-C₄-alkyloxy, hydroxy, —CF₃, —CHF₂, CN, NO₂, fluorine, chlorine or bromine, or
R^{X} and R^{X′} together denote a single bond or a double-bonded group selected from O, S, NH— and CH₂.

Particularly preferred compounds of formula 1.12 are those wherein
X⁻ denotes chloride, bromide or methanesulphonate, preferably bromide;
D and B which may be identical or different, preferably identical, denote S or CH═CH;
R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{X} and R^{X′} which may be identical or different, denote hydrogen, —CF₃ or fluorine, preferably hydrogen, or
R^{X} and R^{X′} together denote a single bond or —O.

Particularly preferred compounds of formula 1.12 are also those wherein
X⁻ denotes bromide;
D and B denotes —CH═CH—;
R¹⁶ denotes hydrogen, hydroxy or methyl;
R^{1″} and R^{2″} denote methyl;
R¹⁷, R¹⁸, R^{17′} and R^{18′}, which may be identical or different, denote hydrogen or fluorine, preferably hydrogen;
R^{X} and R^{X′} which may be identical or different, denote hydrogen or fluorine, preferably hydrogen, or
R^{X} and R^{X′} together denote a single bond or the group —O.

Of particular importance are compounds of formula 1.12 selected from the group comprising:
cyclopropyltropine benzilate methobromide (1.12.1);
cyclopropyltropine 2,2-diphenylpropionate methobromide (1.12.2);
cyclopropyltropine 9-hydroxy-xanthene-9-carboxylate methobromide (1.12.3);
cyclopropyltropine 9-methyl-fluorene-9-carboxylate methobromide (1.12.4);
cyclopropyltropine 9-methyl-xanthene-9-carboxylate methobromide (1.12.5);
cyclopropyltropine 9-hydroxy-fluorene-9-carboxylate methobromide (1.12.6);
methyl cyclopropyltropine 4,4′-difluorobenzilate methobromide (1.12.7).

The compounds of formula 1.12 may optionally be present in the form of their enantiomers, mixtures of their enantiomers or racemates, as well as optionally in the form of their hydrates and/or solvates.

In another preferred embodiment of the present invention compounds of formula 1.13 are used as anticholinergics 1, 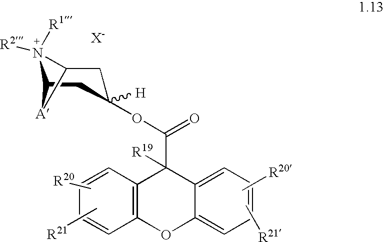
wherein X⁻ may have the meanings given above and wherein
A′ denotes a double-bonded group selected from 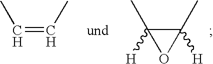
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃—C₆-cycloalkyl, hydroxy or halogen, or
R^{1′″} and R^{2′″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen.

The compounds of formula 1.13 are known in the art (WO 03/064417).

Preferred compounds of formula 1.13 are those wherein
A′ denotes a double-bonded group selected from 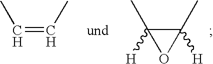
X⁻ denotes chloride, bromide or methanesulphonate, preferably bromide;
R¹⁹ denotes hydroxy or methyl;
R^{1′″} and R^{2′″} which may be identical or different, denote methyl or ethyl, preferably methyl;
R²⁰, R²¹, R^{20′} and R^{21′} which may be identical or different, denote hydrogen, —CF₃, —CHF₂ or fluorine, preferably hydrogen or fluorine.

Particularly preferred compounds of formula 1.13 are those wherein
A′ denotes a double-bonded group selected from 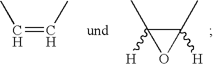
X⁻ denotes bromide;
R¹⁹ denotes hydroxy or methyl, preferably methyl;
R^{1′″} and R^{2′″} which may be identical or different, denote methyl or ethyl, preferably methyl;

R³, R⁴, R^{3′} and R^{4′} which may be identical or different, denote hydrogen or fluorine.

Of particular importance are compounds of formula 1.13, which are selected from the group comprising:
tropenol 9-hydroxy-xanthene-9-carboxylate methobromide (1.13.1);
scopine 9-hydroxy-xanthene-9-carboxylate methobromide (1.13.2);
tropenol 9-methyl-xanthene-9-carboxylate methobromide (1.13.3);
scopine 9-methyl-xanthene-9-carboxylate methobromide (1.13.4);
tropenol 9-ethyl-xanthene-9-carboxylate methobromide (1.13.5);
tropenol 9-difluoromethyl-xanthene-9-carboxylate methobromide (1.13.6);
scopine 9-hydroxymethyl-xanthene-9-carboxylate methobromide (1.13.7).

The compounds of formula 1.13 may optionally be present in the form of their enantiomers, mixtures of their enantiomers or racemates, as well as optionally in the form of their hydrates and/or solvates.

The alkyl groups used, unless otherwise stated, are branched and unbranched alkyl groups having 1 to 4 carbon atoms. Examples include: methyl, ethyl, propyl or butyl. The groups methyl, ethyl, propyl or butyl may optionally also be referred to by the abbreviations Me, Et, Prop or Bu. Unless otherwise stated, the definitions propyl and butyl also include all possible isomeric forms of the groups in question. Thus, for example, propyl includes n-propyl and iso-propyl, butyl includes iso-butyl, sec. butyl and tert.-butyl, etc.

The cycloalkyl groups used, unless otherwise stated, are alicyclic groups with 3 to 6 carbon atoms. These are the cyclopropyl, cyclobutyl, cyclopentyl and cyclohexyl groups. According to the invention cyclopropyl is of particular importance within the scope of the present invention.

The alkylene groups used, unless otherwise stated, are branched and unbranched double-bonded alkyl bridges with 1 to 4 carbon atoms. Examples include: methylene, ethylene, propylene or butylene.

The alkylene-halogen groups used, unless otherwise stated, are branched and unbranched double-bonded alkyl bridges with 1 to 4 carbon atoms which may be mono-, di- or trisubstituted, preferably disubstituted, by a halogen. Accordingly, unless otherwise stated, the term alkylene-OH groups denotes branched and unbranched double-bonded alkyl bridges with 1 to 4 carbon atoms which may be mono-, di- or trisubstituted, preferably monosubstituted, by a hydroxy.

The alkyloxy groups used, unless otherwise stated, are branched and unbranched alkyl groups with 1 to 4 carbon atoms which are linked via an oxygen atom. The following may be mentioned, for example: methyloxy, ethyloxy, propyloxy or butyloxy. The groups methyloxy, ethyloxy, propyloxy or butyloxy may optionally also be referred to by the abbreviations MeO, EtO, PropO or BuO. Unless otherwise stated, the definitions propyloxy and butyloxy also include all possible isomeric forms of the groups in question. Thus, for example, propyloxy includes n-propyloxy and iso-propyloxy, butyloxy includes iso-butyloxy, sec. butyloxy and tert.-butyloxy, etc. The word alkoxy may also possibly be used within the scope of the present invention instead of the word alkyloxy. The groups methyloxy, ethyloxy, propyloxy or butyloxy may optionally also be referred to as methoxy, ethoxy, propoxy or butoxy.

The alkylene-alkyloxy groups used, unless otherwise stated, are branched and unbranched double-bonded alkyl bridges with 1 to 4 carbon atoms which may be mono-, di- or trisubstituted, preferably monosubstituted, by an alkyloxy group.

The —O—CO-alkyl groups used, unless otherwise stated, are branched and unbranched alkyl groups with 1 to 4 carbon atoms which are bonded via an ester group. The alkyl groups are bonded directly to the carbonylcarbon of the ester group. The term —O—CO-alkyl-halogen group should be understood analogously. The group —O—CO—CF₃ denotes trifluoroacetate. Within the scope of the present invention halogen denotes fluorine, chlorine, bromine or iodine. Unless otherwise stated, fluorine and bromine are the preferred halogens. The group CO denotes a carbonyl group.

Within the scope of the present invention any reference to anticholinergics 1′ is to be understood as being a reference to the pharmacologically active cations of the respective salts. These cations are tiotropium (1.1′, oxitropium (1.2′), flutropium (1.3′, ipratropium (1.4′), glycopyrronium (1.5′, trospium (1.6′) as well as the cations shown below:

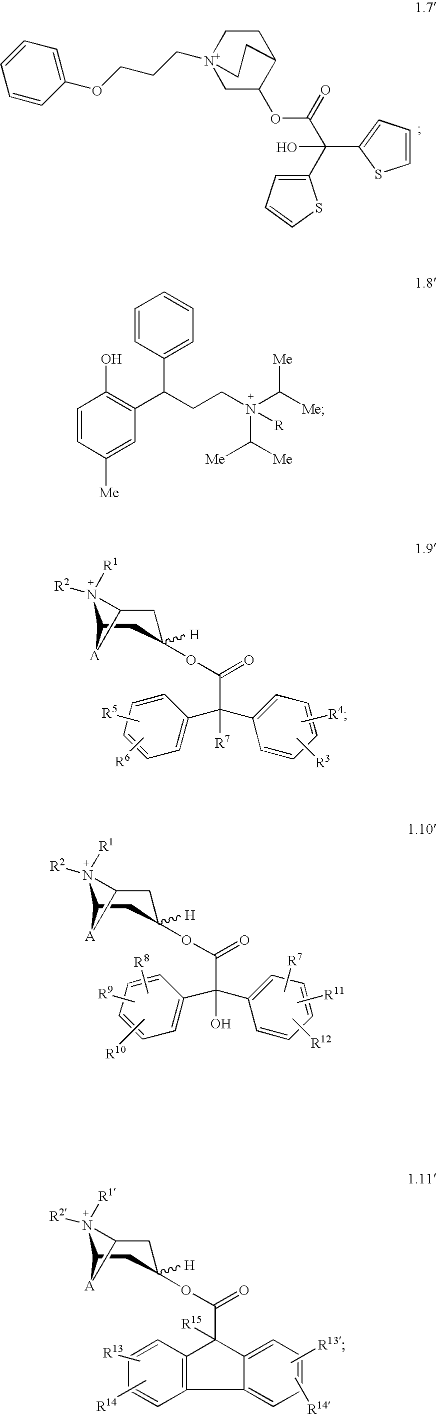

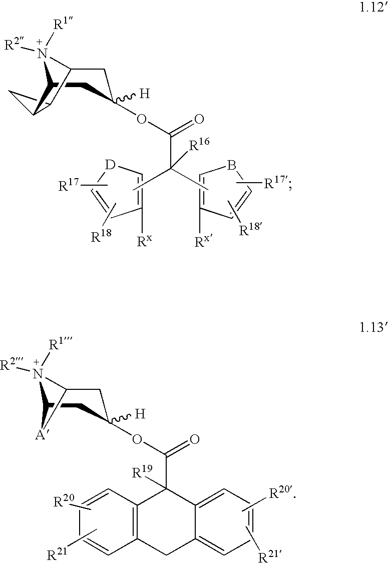

Preferably, the present invention relates to the use of the above-mentioned anticholinergics 1 for preparing a pharmaceutical composition for the prevention or treatment, preferably the treatment, of proliferative processes in cell types selected from among the fibroblasts, myofibroblasts, epithelial cells, endothelial cells, serous and mucosal cells in submucosal glands, Clara cells, type I+II pneumocytes and goblet cells.

Preferably the present invention relates to the above-mentioned use of the above-mentioned anticholinergics 1 for preparing a pharmaceutical composition for the prevention or treatment, preferably the treatment, of proliferative processes which occur in diseases of the upper and lower respiratory organs including the lungs.

Preferably the present invention relates to the above-mentioned use of the above-mentioned anticholinergics 1 for preparing a pharmaceutical composition for the prevention or treatment, preferably the treatment, of proliferative processes which occur in diseases selected from the group consisting of lung inflammation, pulmonary hypertension, pulmonary emphysema, pulmonary fibrosis, pulmonary oedema, bronchiectasis, Adult Respiratory Distress Syndrome (ARDS), Boeck's disease, fibrosing alveolitis, pulmonary embolism, pneumoconiosis (e.g. asbestosis, silicosis), lung cancer and tuberculosis.

Preferably the present invention relates to the above-mentioned use of the above-mentioned anticholinergics 1 for preparing a pharmaceutical composition for the treatment of proliferative processes which occur in diseases selected from the group consisting of pulmonary inflammation, pulmonary hypertension, pulmonary emphysema, pulmonary oedema, Adult Respiratory Distress Syndrome (ARDS), Boeck's disease, pulmonary fibrosis, pulmonary embolism, pneumoconiosis (e.g. asbestosis, silicosis), lung cancer and tuberculosis.

Preferably the present invention relates to the above-mentioned use of the above-mentioned anticholinergics 1 for preparing a pharmaceutical composition for the prevention or treatment, preferably the treatment, of proliferative processes which occur in pulmonary inflammation. Moreover the present invention relates to the use of the above-mentioned anticholinergics 1 for preparing a pharmaceutical composition for the prevention or treatment, preferably for the treatment of pulmonary inflammation.

Preferably the present invention relates to the above-mentioned use of the above-mentioned anticholinergics 1 for preparing a pharmaceutical composition for the prevention or treatment, preferably the treatment, of proliferative processes which occur in pulmonary hypertension. Moreover the present invention relates to the use of the above-mentioned anticholinergics 1 for preparing a pharmaceutical composition for the prevention or treatment, preferably for the treatment of pulmonary hypertension.

Preferably the present invention relates to the above-mentioned use of the above-mentioned anticholinergics 1 for preparing a pharmaceutical composition for the prevention or treatment, preferably the treatment, of proliferative processes which occur in pulmonary emphysema. Moreover the present invention relates to the use of the above-mentioned anticholinergics 1 for preparing a pharmaceutical composition for the prevention or treatment, preferably for the treatment of pulmonary emphysema.

Preferably the present invention relates to the above-mentioned use of the above-mentioned anticholinergics 1 for preparing a pharmaceutical composition for the prevention or treatment, preferably the treatment, of proliferative processes which occur in pulmonary oedemas. Moreover the present invention relates to the use of the above-mentioned anticholinergics 1 for preparing a pharmaceutical composition for the prevention or treatment, preferably for the treatment of pulmonary oedema.

Preferably the present invention relates to the above-mentioned use of the above-mentioned anticholinergics 1 for preparing a pharmaceutical composition for the prevention or treatment, preferably the treatment, of proliferative processes which occur in ARDS. Moreover the present invention relates to the use of the above-mentioned anticholinergics 1 for preparing a pharmaceutical composition for the prevention or treatment, preferably for the treatment of ARDS.

Preferably the present invention relates to the above-mentioned use of the above-mentioned anticholinergics 1 for preparing a pharmaceutical composition for the prevention or treatment, preferably the treatment, of proliferative processes which occur in Boeck's disease. Moreover the present invention relates to the use of the above-mentioned anticholinergics 1 for preparing a pharmaceutical composition for the prevention or treatment, preferably for the treatment of Boeck's disease.

Preferably the present invention relates to the above-mentioned use of the above-mentioned anticholinergics 1 for preparing a pharmaceutical composition for the prevention or treatment, preferably the treatment, of proliferative processes which occur in pulmonary fibrosis. Moreover the present invention relates to the use of the above-mentioned anticholinergics 1 for preparing a pharmaceutical composition for the prevention or treatment, preferably for the treatment of pulmonary fibrosis.

Preferably the present invention relates to the above-mentioned use of the above-mentioned anticholinergics 1 for preparing a pharmaceutical composition for the prevention or treatment, preferably the treatment, of proliferative processes which occur in pulmonary embolism. Moreover the present invention relates to the use of the above-mentioned anticholinergics 1 for preparing a pharmaceutical composition for the prevention or treatment, preferably for the treatment of pulmonary embolism.

Preferably the present invention relates to the above-mentioned use of the above-mentioned anticholinergics 1 for preparing a pharmaceutical composition for the prevention or treatment, preferably the treatment, of proliferative processes which occur in pneumoconiosis (e.g. asbestosis, silicosis). Moreover the present invention relates to the use of the above-mentioned anticholinergics 1 for preparing a pharmaceutical composition for the prevention or treatment, preferably for the treatment of pneumoconiosis (e.g. asbestosis, silicosis).

Preferably the present invention relates to the above-mentioned use of the above-mentioned anticholinergics 1 for preparing a pharmaceutical composition for the prevention or treatment, preferably the treatment, of proliferative processes which occur in lung cancer. Moreover the present invention relates to the use of the above-mentioned anticholinergics 1 for preparing a pharmaceutical composition for the prevention or treatment, preferably for the treatment of lung cancer.

Preferably the present invention relates to the above-mentioned use of the above-mentioned anticholinergics 1 for preparing a pharmaceutical composition for the prevention or treatment, preferably the treatment, of proliferative processes which occur in tuberculosis. Moreover the present invention relates to the use of the above-mentioned anticholinergics 1 for preparing a pharmaceutical composition for the prevention or treatment, preferably for the treatment of tuberculosis.

In another aspect the present invention relates to pharmaceutical formulations containing an anticholinergic 1 for the treatment of the above-mentioned diseases. Moreover the present invention relates to the use of formulations containing an anticholinergic 1 for preparing a pharmaceutical composition for the prevention or treatment, preferably for the treatment of the above-mentioned diseases.

Pharmaceutical formulations, without restricting the scope of the invention thereto, may in the case of tiotropium 1.1′ contain amounts of anticholinergic (1.1′) such that each individual dose contains 0.1-80 μg, preferably 0.5-60 μg, particularly preferably about 1 to 50 μg. For example and without restricting the scope of the invention thereto, 2.5 μg, 5 μg, 10 μg, 18 μg, 20 μg, 36 μg or 40 μg of 1.1′ may be administered in each individual dose. The corresponding amount of salt 1.1 used or optionally the hydrate or solvate used can easily be calculated by the skilled man, depending on the choice of anion. If tiotropium bromide is used, for example, as the preferred tiotropium salt 1.1 according to the invention, the amounts of active substance of 1.1′ administered in each single dose correspond to the following amounts of 1.1 administered in each single dose: 3 μg, 6 μg, 12 μg, 21.7 μg, 24.1 μg, 43.3 μg and 48.1 μof 1.1. In the case of tiotropium 1.1′ the above-mentioned dosages are preferably administered once or twice a day, while once-a-day administration is particularly preferred according to the invention.

Pharmaceutical formulations, without restricting the scope of the invention thereto, may in the case of the cation 1.2′ contain amounts of anticholinergic (1.2′) such that each individual dose contains 1-500 μg, preferably 5-300 μg, particularly preferably 15-200 μg. For example and without restricting the scope of the invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1.2′ may be administered in each individual dose. The corresponding amount of salt 1.2 used or optionally the hydrate or solvate used can easily be calculated by the skilled man, depending on the choice of anion. In the case of oxitropium 1.2′ the dosages specified above are preferably administered one to four times a day, while administration two to three times a day is particularly preferred according to the invention.

Pharmaceutical formulations, without restricting the scope of the invention thereto, may in the case of the cation 1.3′ contain amounts of anticholinergic (1.3′) such that each individual dose contains 1-500 μg, preferably 5-300 μg, particularly preferably 15-200 μg. For example and without restricting the scope of the invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1.3′ may be administered in each individual dose. The corresponding amount of salt 1.3 used or optionally the hydrate or solvate used can easily be calculated by the skilled man, depending on the choice of anion. In the case of flutropium 1.3′ the dosages specified above are preferably administered one to four times a day, while administration two to three times a day is particularly preferred according to the invention.

Pharmaceutical formulations, without restricting the scope of the invention thereto, may in the case of the cation 1.4′ contain amounts of anticholinergic (1.4′) such that each individual dose contains 1-500 μg, preferably 5-300 μg, particularly preferably 20-200 μg. For example and without restricting the scope of the invention thereto, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1.4′ may be administered in each individual dose. The corresponding amount of salt 1.4 used or optionally the hydrate or solvate used can easily be calculated by the skilled man, depending on the choice of anion. In the case of ipratropium 1.4′ the dosages specified above are preferably administered one to four times a day, while administration two to three times a day and most preferably three times a day is particularly preferred according to the invention.

Pharmaceutical formulations, without restricting the scope of the invention thereto, may in the case of the cation 1.5′ contain amounts of anticholinergic (1.5′) such that each individual dose contains 1-500 μg, preferably 5-300 μg, particularly preferably 15-200 μg. For example and without restricting the scope of the invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1.5′ may be administered in each individual dose. The corresponding amount of salt 1.5 used or optionally the hydrate or solvate used can easily be calculated by the skilled man, depending on the choice of anion. In the case of glycopyrronium 1.5′ the dosages specified above are preferably administered one to four times a day, while administration two to three times a day is particularly preferred according to the invention.

Pharmaceutical formulations, without restricting the scope of the invention thereto, may in the case of the cation 1.6′ contain amounts of anticholinergic (1.6′) such that each individual dose contains 1000-6500 μg, preferably 2000-6000 μg, particularly preferably 3000-5500 μg, particularly preferably 4000-5000 μg. For example and without restricting the scope of the invention thereto, 3500 μg, 3750 μg, 4000 μg, 4250 μg, 4500 μg, 4750 μg, or 5000 μg of 1.6′ may be administered in each individual dose. The corresponding amount of salt 1.6 used or optionally the hydrate or solvate used can easily be calculated by the skilled man, depending on the choice of anion. In the case of trospium 1.6′ the dosages specified above are preferably administered one to four times a day, while administration two to three times a day is particularly preferred according to the invention.

Pharmaceutical formulations, without restricting the scope of the invention thereto, may in the case of the cation 1.7′ contain amounts of anticholinergic (1.7′) such that each individual dose contains 50-1000 μg, preferably 100-800 μg, particularly preferably 200-700 μg, particularly preferably 300-600 μg. For example and without restricting the scope of the invention thereto, 300 μg, 350 μg, 400 μg, 450 μg, 500 μg, 550 μg, or 600 μg of 1.7′ may be administered in each individual dose. The corresponding amount of salt 1.7 used or optionally the hydrate or solvate used can easily be calculated by the skilled man, depending on the choice of anion. In the case of the cation 1.7′ the dosages specified above are preferably administered one to three times a day, while administration once or twice a day, most preferably once a day, is particularly preferred according to the invention.

Pharmaceutical formulations, without restricting the scope of the invention thereto, may in the case of the cations 1.9′ and 1.10′ contain amounts of anticholinergic (1.9′ or 1.10′) such that each individual dose contains 1-500 μg, preferably 5-300 μg, particularly preferably 15-200 μg. For example and without restricting the scope of the invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1.9′ or 1.10′ may be administered in each individual dose. The corresponding amount of salt 1.9′ or 1.10′ used or optionally the hydrate or solvate used can easily be calculated by the skilled man, depending on the choice of anion. In the case of the cations 1.9′ or 1.10′ the dosages specified above are preferably administered one to three times a day, while administration once or twice a day, most preferably once a day, is particularly preferred according to the invention.

Pharmaceutical formulations, without restricting the scope of the invention thereto, may in the case of the cations 1.11′ to 1.13′ contain amounts of anticholinergic (1.11′, 1.12′ or 1.13′) such that each individual dose contains 1-500 μg, preferably 5-300 μg, particularly preferably 10-200 μg. For example and without restricting the scope of the invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 1.11′, 1.12′ or 1.13′ may be administered in each individual dose. The corresponding amount of salt 1.11′, 1.12′ or 1.13′ used or optionally the hydrate or solvate used can easily be calculated by the skilled man, depending on the choice of anion. In the case of the cations 1.11, 1.12 or 1.13 the dosages specified above are preferably administered one to three times a day, while administration once or twice a day, most preferably once a day, is particularly preferred according to the invention.

The anticholinergics 1 are preferably administered within the scope of the use according to the invention by inhalation. For this the anticholinergics 1 have to be formulated as inhalable preparations. Inhalable preparations include inhalable powders, propellant-containing metered-dose aerosols or propellant-free inhalable solutions.

Inhalable powders according to the invention optionally contain the anticholinergics 1 in admixture with physiologically acceptable excipients. Within the scope of the present invention, the term propellant-free inhalable solutions also includes concentrates or sterile inhalable solutions ready for use. The formulations which may be used within the scope of the present invention are described in more detail in the next part of the specification.

A) Inhalable Powder:

The inhalable powders which may be used according to the invention may contain 1 either on their own or in admixture with suitable physiologically acceptable excipients.

If the anticholinergics 1 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art.

Inhalable powders according to the invention which contain a physiologically acceptable excipient in addition to 1 may be administered, for example, by means of inhalers which deliver a single dose from a supply using a measuring chamber as described in US 4570630A, or by other means as described in DE 36 25 685 A. The inhalable powders according to the invention which contain 1 optionally in conjunction with a physiologically acceptable excipient may be administered, for example, using the inhaler known by the name Turbuhaler® or using inhalers as disclosed for example in EP 237507 A. Preferably, the inhalable powders according to the invention which contain physiologically acceptable excipient in addition to 1 are packed into capsules (to produce so-called inhalettes) which are used in inhalers as described, for example, in WO 94/28958.

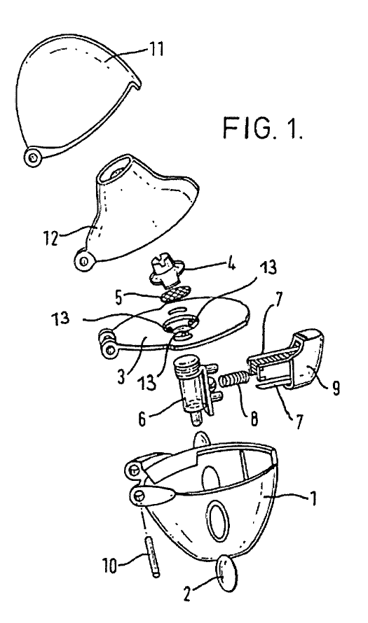

A particularly preferred inhaler for using the pharmaceutical combination according to the invention in inhalettes is shown in FIG. 1.

This inhaler (Handyhaler) for inhaling powdered pharmaceutical compositions from capsules is characterised by a housing 1 containing two windows 2, a deck 3 in which there are air inlet ports and which is provided with a screen 5 secured by means of a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and airholes 13 for adjusting the flow resistance.

If the inhalable powders according to the invention are packed into capsules (inhalettes) for the preferred use described above, the quantities packed into each capsule should be 1 to 30 mg, preferably 3 to 20 mg, preferably 5 to 10 mg of inhalable powder per capsule. These capsules contain, according to the invention, the doses of the cations 1 mentioned hereinbefore for each single dose.

B) Propellant Gas-Driven Inhalation Aerosols:

Inhalation aerosols containing propellant gas which may be used according to the invention may contain 1 dissolved in the propellant gas or in dispersed form. The propellant gases which may be used to prepare the inhalation aerosols are known from the prior art. Suitable propellant gases are selected from among hydrocarbons such as n-propane, n-butane or isobutane and halohydrocarbons such as preferably fluorinated derivatives of methane, ethane, propane, butane, cyclopropane or cyclobutane. The propellant gases mentioned above may be used on their own or in mixtures thereof. Particularly preferred propellant gases are fluorinated alkane derivatives selected from TG134a (1,1,1,2-tetrafluoroethane), TG227 (1,1,1,2,3,3,3-heptafluoropropane) and mixtures thereof.

The propellant-driven inhalation aerosols which may be used according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1. The propellant-containing inhalation aerosol which may be used within the scope of the use according to the invention contain, for example, 0.002 to 5 wt.-%, 0.01 to 3 wt.-%, 0.015 to 2 wt.-% of active substance 1.

If the active substances 1 are present in dispersed formn, the particles of active substance preferably have an average particle size of up to 10 μm, preferably from 0.1 to 5 μm, more preferably from 1 to 5 μm.

The propellant-driven inhalation aerosols which may be used according to the invention may be administered using inhalers known in the art (MDIs=metered dose inhalers). Accordingly, in another aspect, the present invention relates to the novel use of 1 for preparing pharmaceutical compositions in the form of propellant-driven aerosols as hereinbefore described combined with one or more inhalers suitable for administering these aerosols.

The present invention further relates to the novel use of 1 for the manufacture of cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions:

Tiotropium salts 1 are preferably used according to the invention for preparing propellant-free inhalable solutions and inhalable suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 are adjusted to a pH of 2 to 7, preferably 2 to 5, using suitable acids. The pH may be adjusted using acids selected from inorganic or organic acids. Examples of suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium edetate, as stabiliser or complexing agent can be omitted. Other embodiments may contain this compound or these compounds.

In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

The preferred propellant-free inhalable solutions which may be used within the scope of the use according to the invention contain only benzalkonium chloride and sodium edetate, in addition to the solvent water and the tiotropium salts 1. In another preferred embodiment, no sodium edetate is present.

The propellant-free inhalable solutions which may be used according to the invention are administered in particular using inhalers of the kind which are capable of nebulising a small amount of a liquid formulation in the therapeutic dosage within a few seconds to produce an aerosol suitable for therapeutic inhalation. Within the scope of the present invention, preferred nebulisers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are also known by the name Respimat®.

This nebuliser (Respimat®) can advantageously be used to produce the inhalable aerosols according to the invention containing the tiotropium salts 1. Because of its cylindrical shape and handy size of less than 9 to 15 cm long and 2 to 4 cm wide, this device can be carried at all times by the patient. The nebuliser sprays a defined volume of the pharmaceutical formulation at high pressures through small nozzles so as to produce inhalable aerosols.

The preferred atomiser essentially consists of an upper housing part, a pump housing, a nozzle, a locking mechanism, a spring housing, a spring and a storage container, characterised by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 Mpa (about 50 to 600 bar), preferably 10 to 60 Mpa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the nozzle body.

The nozzle in the nozzle body is preferably microstructured, i.e. produced by microtechnology. Microstructured valve bodies are disclosed for example in WO-94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns. In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by pressing gently on the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to which reference is hereby made.

The nebuliser described above is suitable for nebulising the aerosol preparations according to the invention to produce an aerosol suitable for inhalation.

If the formulation according to the invention is nebulised using the technology described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the propellant-free inhalable solutions which may be used within the scope of the use according to the invention may also be nebulised by means of inhalers other than those described above, e.g. jet stream inhalers or other stationary nebulisers.

Accordingly, in a further aspect, the invention relates to the use according to the invention of anticholinergics 1 for preparing a pharmaceutical formulation in the form of propellant-free inhalable solutions or suspensions as described above combined with a device suitable for administering these formulations, preferably in conjunction with the Respimat®. Preferably, the invention relates to the use of 1 within the scope of the invention for preparing propellant-free inhalable solutions or suspensions characterised in that they contain 1 in conjunction with the device known by the name Respimat®. In addition, the present invention relates to the use according to the invention of the above-mentioned devices for inhalation, preferably the Respimat®, characterised in that they contain the propellant-free inhalable solutions or suspensions according to the invention as described hereinbefore.

The propellant-free inhalable solutions or suspensions which may be used within the scope of the use of 1 according to the invention may take the form of concentrates or sterile inhalable solutions or suspensions ready for use, as well as the above-mentioned solutions and suspensions designed for use in a Respimat®. Formulations ready for use may be produced from the concentrates, for example, by the addition of isotonic saline solutions. Sterile formulations ready for use may be administered using powered fixed or portable nebulisers which produce inhalable aerosols by means of ultrasound or compressed air by the Venturi principle or other principles.

Accordingly, in another aspect, the present invention relates to the use of 1 according to the invention in the form of propellant-free inhalable solutions or suspensions as described hereinbefore which take the form of concentrates or sterile formulations ready for use, combined with a device suitable for administering these solutions, characterised in that the device is a powered free-standing or portable nebuliser which produces inhalable aerosols by means of ultrasound or compressed air by the Venturi principle or other methods.

The Examples which follow serve to illustrate the present invention in more detail without 5 restricting the scope of the invention to the following embodiments by way of example.

### EXAMPLES OF FORMULATIONS

A) Inhalable powder:

| | |
|---|---|
| ingredients | μg per capsule |

1)

| | |
|---|---|
| 1.1-bromide | 10.8 |
| lactose | 4989.2 |
| total | 5000 |

2)

| | |
|---|---|
| 1.1-bromide | 21.7 |
| lactose | 4978.3 |
| total | 5000 |

3)

| | |
|---|---|
| 1.1-bromide × H₂O | 22.5 |
| lactose | 4977.5 |
| total | 5000 |

4)

| | |
|---|---|
| 1.4-bromide | 200 |
| trehalose | 12300 |
| total | 12500 |

5)

| | |
|---|---|
| 1.4-bromide | 200 |
| lactose | 12300 |
| total | 12500 |

6)

| | |
|---|---|
| 1.7-bromide | 300 |
| lactose | 4700 |
| total | 5000 |

7)

| | |
|---|---|
| 1.7-bromide | 600 |
| lactose | 4400 |
| total | 5000 |

8)

| | |
|---|---|
| 1.7-bromide | 150 |
| lactose | 4850 |
| total | 5000 |

9)

| | |
|---|---|
| 1.8-base | 50 |
| lactose | 4950 |
| total | 5000 |

10)

| | |
|---|---|
| 1.8-base | 150 |
| lactose | 4850 |
| total | 5000 |

11)

| | |
|---|---|
| 1.8-base | 300 |
| lactose | 4700 |
| total | 5000 |

12)

| | |
|---|---|
| 1.5-bromide | 15 |
| lactose | 4985 |
| total | 5000 |

13)

| | |
|---|---|
| 1.5-bromide | 30 |
| lactose | 4970 |
| total | 5000 |

14)

| | |
|---|---|
| 1.5-bromide | 60 |
| lactose | 4940 |
| total | 5000 |

15)

| | |
|---|---|
| 1.9.2 | 200 |
| lactose | 12300 |
| total | 12500 |

16)

| | |
|---|---|
| 1.9.2 | 100 |
| lactose | 12400 |
| total | 12500 |

17)

| | |
|---|---|
| 1.9.1 | 200 |
| trehalose | 12300 |
| total | 15000 |

18)

| | |
|---|---|
| 1.9.1 | 200 |
| lactose | 12300 |
| total | 15000 |

19)

| | |
|---|---|
| 1.9.2 | 100 |
| maltose | 12400 |
| total | 12500 |

20)

| | |
|---|---|
| 1.9.1 | 200 |
| trehalose | 12300 |
| total | 12500 |

21)

| | |
|---|---|
| 1.9.2 | 100 |
| lactose | 4900 |
| total | 5000 |

22)

| | |
|---|---|
| 1.11.6 | 200 |
| lactose | 12300 |
| total | 15000 |

23)

| | |
|---|---|
| 1.11.6 | 100 |
| lactose | 12400 |
| total | 12500 |

24)

| | |
|---|---|
| 1.11.5 | 200 |
| trehalose | 12300 |
| total | 15000 |

25)

| | |
|---|---|
| 1.11.5 | 200 |
| lactose | 12300 |
| total | 15000 |

26)

| | |
|---|---|
| 1.11.6 | 100 |
| maltose | 12400 |
| total | 12500 |

27)

| | |
|---|---|
| 1.11.5 | 200 |
| trehalose | 12300 |
| total | 12500 |

28)

| | |
|---|---|
| 1.11.6 | 100 |
| lactose | 4900 |
| total | 5000 |

B) Propellant-Containing Inhalable Aerosols:

| | |
|---|---|
| ingredients | % by weight |

1)

| | |
|---|---|
| 1.1-bromide | 0.015 |
| soya lecithin | 0.2 |
| TG 134a:TG227 = 2:3 | ad 100 |

2)

| | |
|---|---|
| 1.1-bromide | 0.029 |
| absolute ethanol | 0.5 |
| isopropyl myristate | 0.1 |
| TG 227 | ad 100 |

3)

| | |
|---|---|
| 1.1-bromide | 0.042 |
| absolute ethanol | 30 |
| purified water | 1.5 |
| anhydrous citric acid | 0.002 |
| TG 134a | ad 100 |

4)

| | |
|---|---|
| 1.5-bromide | 0.045 |
| soya lecithin | 0.2 |
| TG 134a:TG227 = 2:3 | ad 100 |

5)

| | |
|---|---|
| 1.5-bromide | 0.09 |
| absolute ethanol | 0.5 |
| isopropyl myristate | 0.1 |
| TG 227 | ad 100 |

6)

| | |
|---|---|
| 1.5-bromide | 0.12 |
| ethanol | 30 |
| purified water | 1.5 |
| anhydrous citric acid | 0.002 |
| TG 134a | ad 100 |

7)

| | |
|---|---|
| 1.9.1 | 0.2 |
| ethanol | 25 |
| purified water | 1.0 |
| anhydrous citric acid | 0.003 |
| TG 134a | ad 100 |

8)

| | |
|---|---|
| 1.9.1 | 0.1 |
| ethanol | 20.0 |
| aq. HCl (0.01 mol/l) | 2.0 |
| TG 134a | ad 100 |

9)

| | |
|---|---|
| 1.9.1 | 0.1 |
| ethanol | 15.0 |
| purified water | 2.0 |
| anhydrous citric acid | 0.004 |
| TG 227 | ad 100 |

10)

| | |
|---|---|
| 1.9.2 | 0.2 |
| ethanol | 25 |
| purified water | 1.0 |
| anhydrous citric acid | 0.003 |
| TG 134a | ad 100 |

11)

| | |
|---|---|
| 1.9.2 | 0.1 |
| ethanol | 20.0 |
| aq. HCl (0.01 mol/l) | 2.0 |
| TG 134a | ad 100 |

12)

| | |
|---|---|
| 1.9.2 | 0.1 |
| ethanol | 15.0 |
| purified water | 2.0 |
| anhydrous citric acid | 0.004 |
| TG 227 | ad 100 |

13)

| | |
|---|---|
| 1.9.2 | 0.2 |
| ethanol | 30.0 |
| purified water | 1.0 |
| ascorbic acid | 0.005 |
| TG 134a | ad 100 |

14)

| | |
|---|---|
| 1.9.2 | 0.05 |
| ethanol | 40.0 |
| anhydrous citric acid | 0.004 |
| TG 227 | ad 100 |

C) Propellant-Free Inhalable Solutions:

| | |
|---|---|
| ingredients | mg/100 mL |

1) Solution for use in the Respimat ®:

| | |
|---|---|
| 1.1-bromide | 148.5 |
| benzalkonium chloride | 10 |
| sodium edetate | 10 |
| hydrochloric acid (aq) | ad pH 2.9 |
| water | ad 100 mL |

2) Solution for use in the Respimat ®:

| | |
|---|---|
| 1.1-bromide | 148.5 |
| benzalkonium chloride | 10 |
| hydrochloric acid (aq) | ad pH 2.9 |
| water | ad 100 mL |

3) Solution for use in the Respimat ®:

| | |
|---|---|
| 1.1-bromide | 297.1 |
| benzalkonium chloride | 10 |
| sodium edetate | 10 |
| hydrochloric acid (aq) | ad pH 2.9 |
| water | ad 100 mL |

4) Solution for use in the Respimat ®:

| | |
|---|---|
| 1.1-bromide | 297.1 |
| benzalkonium chloride | 10 |
| hydrochloric acid (aq) | ad pH 2.9 |
| water | ad 100 mL |

5) Solution for use in the Respimat ®:

| | |
|---|---|
| 1.1-bromide | 148.5 |
| benzalkonium chloride | 8 |
| sodium edetate | 50 |
| hydrochloric acid (aq) | ad pH 2.5 |
| water | ad 100 mL |

6) Solution for use in the Respimat ®:

| | |
|---|---|
| 1.1-bromide | 1.5 |
| benzalkonium chloride | 8 |
| sodium edetate | 10 |
| hydrochloric acid (aq) | ad pH 2.5 |
| water | ad 100 mL |

7) Solution for use in the Respimat ®:

| | |
|---|---|
| 1.1-bromide | 14.9 |
| benzalkonium chloride | 10 |
| sodium edetate | 50 |
| hydrochloric acid (aq) | ad pH 3.5 |
| water | ad 100 mL |

8) Concentrated solution:

| | |
|---|---|
| 1.1-bromide | 1486.1 |
| benzalkonium chloride | 20 |
| sodium edetate | 100 |
| hydrochloric acid (aq) | ad pH 3.5 |
| water | ad 100 mL |

9) Solution for use in the Respimat ®:

| | |
|---|---|
| 1.5-bromide | 445.5 |
| benzalkonium chloride | 10 |
| sodium edetate | 10 |
| hydrochloric acid (aq) | ad pH 2.9 |
| water | ad 100 mL |

10) Solution for use in the Respimat ®:

| | |
|---|---|
| 1.5-bromide | 445.5 |
| benzalkonium chloride | 10 |
| hydrochloric acid (aq) | ad pH 2.9 |
| water | ad 100 mL |

1) a method of treating proliferative processes comprising administering to a patient in need thereof a pharmaceutically effective amount of an anticholinergic 1 and/or a pharmaceutically acceptable salt or excipient thereof:

2) The method according to claim 1, wherein the proliferative processes are processes in cell types selected from the group consisting of fibroblasts, myofibroblasts, epithelial cells, endothelial cells, serous and mucosal cells in submucosal glands, Clara cells, type I+II pneumocytes and goblet cells.

3) The method according to claim 1, wherein the proliferative processes occur in diseases of the upper and lower respiratory organs including the lungs.

4) The method according to claim 3, wherein the diseases of the upper and lower respiratory organs including the lungs are diseases which are selected from the group comprising pulmonary inflammation, pulmonary hypertension, pulmonary emphysema, pulmonary fibrosis, pulmonary oedema, bronchiectasis, Adult Respiratory Distress Syndrome (ARDS), Boeck's disease, fibrosing alveolitis, pulmonary embolism, pneumoconiosis, lung cancer and tuberculosis.

5) The method according to claim 4, wherein the diseases of the upper and lower respiratory organs including the lungs are diseases which are selected from the group comprising pulmonary inflammation, pulmonary hypertension, pulmonary emphysema, pulmonary oedema, Adult Respiratory Distress Syndrome (ARDS), Boeck's disease, pulmonary fibrosis, pulmonary embolism, pneumoconiosis, lung cancer and tuberculosis.

6) The method according to claim 1, wherein the anticholinergic 1 is a compound selected from the group consisting of tiotropium salts (1.1), oxitropium salts (1.2), flutropium salts (1.3, ipratropium salts (1.4), glycopyrronium salts (1.5) and trospium salts (1.6), optionally in the form of the racemates, enantiomers or hydrates thereof.

7) The method according to claim 1, wherein the anticholinergic 1 is a compound of formula 1.7, 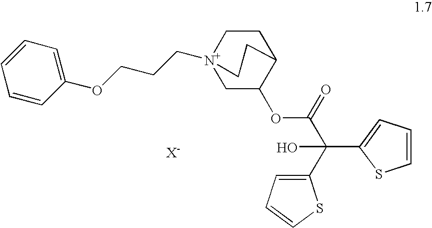
wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
optionally in the form of the racemates, enantiomers or hydrates thereof.

8) The method according to claim 1, wherein the anticholinergic 1 is a compound of formula 1.8, 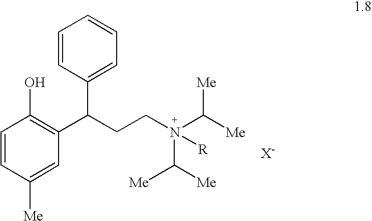
wherein R denotes either methyl (1.8.1) or ethyl (1.8.2) and wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
optionally in the form of the racemates, enantiomers or hydrates thereof.

9) The method according to claim 1, wherein the anticholinergic 1 is a compound of formula 1.9, 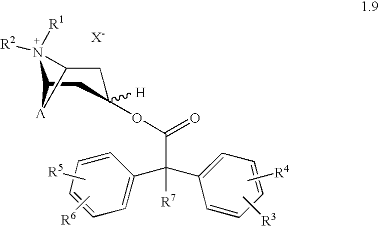
wherein
A denotes a double-bonded group selected from the groups 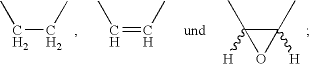
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, CH₂CH₂—OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine, optionally in the form of the racemates, enantiomers or hydrates thereof.

10) The method according to claim 1, wherein the anticholinergic 1 is a compound of formula 1.10, 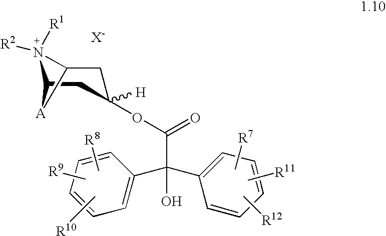
wherein
A denotes a double-bonded group selected from the groups 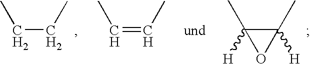
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
R¹ and R² which may be identical or different denote a group selected from methyl, ethyl, n-propyl and iso-propyl, which may optionally be substituted by hydroxy or fluorine, preferably unsubstituted methyl;
R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen,
optionally in the form of the racemates, enantiomers or hydrates thereof.

11) The method according to claim 1, wherein the anticholinergic 1 is a compound of formula 1.11, 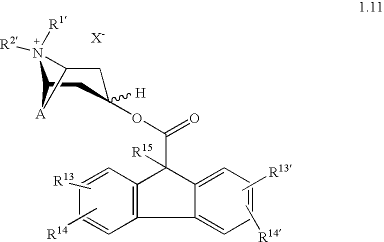
wherein
A denotes a double-bonded group selected from the groups 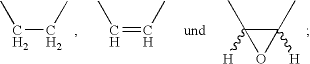
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen, or
R^{1′} and R^{2′} together denote a —C₃-C₅-alkylene bridge;
R¹³, R¹⁴, R^{13′} and R^{14′} which may be identical or different, denote hydrogen, —C₁-C₄-alkyl, —C₁-C₄-alkyloxy, hydroxy, —CF₃, —CHF₂, CN, NO₂ or halogen,
optionally in the form of the racemates, enantiomers or hydrates thereof.

12) The method according to claim 1, wherein the anticholinergic 1 is a compound of formula 1.12, 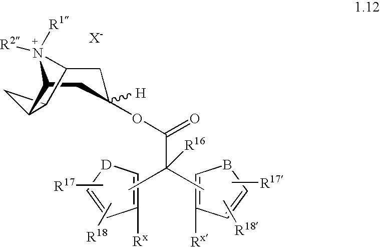
wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
D and B which may be identical or different, preferably identical, denote O, S, NH, CH₂, CH═CH or —N(C₁-C₄-alkyl)-;
R¹⁶ denotes hydrogen, hydroxy, —C₁-C₄-alkyl, —C₁-C₄-alkyloxy, —C₁-C₄-alkylene-halogen, —O—C1-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R1″ and R^{2″} which may be identical or different, denote —C₁-C₅-alkyl, which may optionally be substituted by —C₃-C₆-cycloalkyl, hydroxy or halogen, or
R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{X} and R^{X′} which may be identical or different, denote hydrogen, —C₁-C₄-alkyl, —C₁-C₄-alkyloxy, hydroxy, —CF₃, —CHF₂, CN, NO₂ or halogen, or
R^{X} and R^{X′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂, optionally in the form of the racemates, enantiomers or hydrates thereof.

13) The method according to claim 1, wherein the anticholinergic 1 is a compound of formula 1.13, 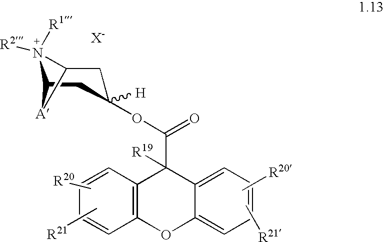
wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
A′ denotes a double-bonded group selected from 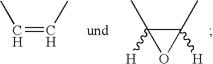
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen, or
R^{1′″} and R^{2′″} together denote a —C₃-C₅-alkylene bridge;
R²⁰, R²¹, R²⁰′ and R²¹′, which may be identical or different, denote hydrogen, —C₁-C₄-alkyl, —C₁-C₄-alkyloxy, hydroxy, —CF₃, —CHF₂, CN, NO₂ or halogen,
optionally in the form of the racemates, enantiomers or hydrates thereof.
